(19) United States
(12) Patent Application Publication
(10) Pub. No.: US 2025/0002466 A1
(43) Pub. Date: Jan. 2, 2025

(54) PROCESS FOR THE PREPARATION OF CENOBAMATE ANND INTERMEDIATES THEREOF

(71) Applicant: METROCHEM API PVT LTD, Hyderabad (IN)

(72) Inventors: Venkateswara Rao NANDEPU, Hyderabad (IN); Hardev NANDEPU, Hyderabad (IN); Narender Rao SOMISETTI, Hyderabad (IN); Venkata Rao BADISA, Hyderabad (IN)

(73) Assignee: METROCHEM API PVT LTD, (omitted)

(21) Appl. No.: 18/837,729

(22) PCT Filed: Feb. 12, 2023
(86) PCT No.: PCT/IB2023/051240

§ 371 Date: Aug. 12, 2024

(30) Foreign Application Priority Data
Feb. 12, 2022 (IN) 202241007524
May 12, 2022 (IN) 202241027288

(51) Int. Cl. C07D 257/04; C07C 29/159; C07C 67/08; C07F 7/18

(52) U.S. Cl. CPC C07D 257/04; C07C 29/159; C07C 67/08; C07F 7/1804

(57) ABSTRACT

The present invention relates to a process for the preparation of Cenobamate and intermediates thereof.

### RELATED APPLICATIONS

This application claims the benefit under Indian Provisional Application No's. 202241007524, filed on Feb. 12, 2022 and 202241027288, filed on May 12, 2022, the contents of which are incorporated by reference herein.

### FIELD OF THE INVENTION

The present invention relates to a process for the preparation of Cenobamate and intermediates thereof.

### BACKGROUND OF THE INVENTION

Cenobamate is a novel new antiepileptic drug, which can be used in the treatment of disorders of the central nervous system, especially as anxiety, depression, convulsion, epilepsy, migraine, bipolar disorder, drug abuse, smoking, ADHD, obesity, sleep disorder, neuropathic pain, stroke, cognitive impairment, neurodegeneration, stroke and muscle spasm.

Cenobamate is chemically known as [(1R)-1-(2-Chlorophenyl)-2-(tetrazol-2-yl) ethyl] carbamate and is structurally represented by the following formula I.

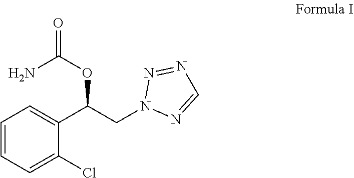 [SMILES: NC(=O)O[C@@H](Cn1ncnn1)c1ccccc1Cl]

Cenobamate was approved in US under the brand name Xcopri. Cenobamate and process for its preparation was first disclosed in U.S. Pat. No. 7,598,279 (the U.S. Pat. No. '279 patent) and the disclosed process is schematically depicted in the following scheme:

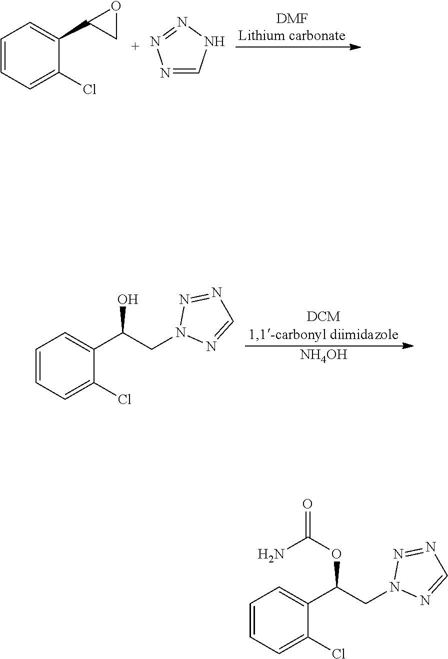

The process reported in US '945 involves reaction of (R)-2-chlorostyrene oxide with 1H-tetrazole in lithium carbonate provides (R)-configured alcohol compound, which on reaction with 1,1′-carbonyl diimidazole in methylene chloride yields Cenobamate. The said process involves column chromatography purification for the preparation of Cenobamate, and not be suitable for large scale production.

The U.S. Pat. No. 8,501,436 patent (the US '436 patent) discloses a preparation method of carbamic acid (R)-1-aryl-2-tetrazolyl-ethyl ester (I), comprising the asymmetric reduction of arylketone (2), followed by carbamation of alcohol (3); the disclosed process is schematically depicted in the following scheme:

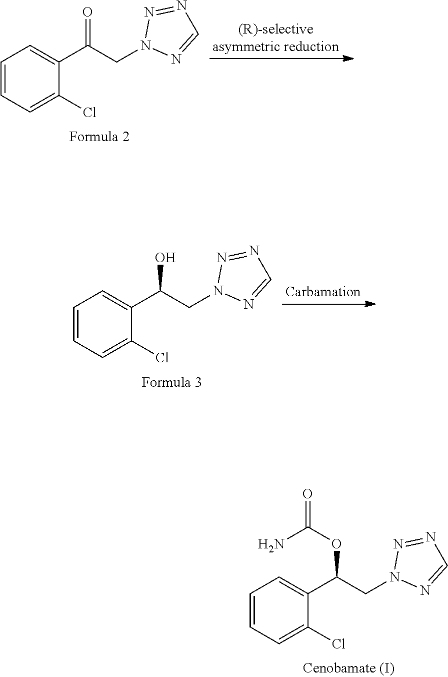 [SMILES: O=C(Cn1ncnn1)c1ccccc1Cl ; O[C@@H](Cn1ncnn1)c1ccccc1Cl ; NC(=O)O[C@@H](Cn1ncnn1)c1ccccc1Cl]

However, there is always a need for an alternative process, which for example, involves use of reagents/solvents that are less expensive and easier to handle, consume smaller amounts of solvents, and provide a higher yield of product with higher purity. Hence, the main objective of the present invention is to provide cost effective and commercially viable process for the preparation of Cenobamate of Formula I.

### SUMMARY OF THE INVENTION

Accordingly, the present invention provides a novel process for the preparation of Cenobamate of formula 1, which is cost effective and commercially viable.

In one aspect, the present invention provides a process for the preparation of Cenobamate of formula I, which comprises:
a) reacting (R)-(−)-2-chloromandelic acid of formula II with an alcohol in the presence of a catalyst to provide a compound of formula III;
b) optionally, reacting the compound of formula III with a suitable hydroxy protecting agent to provide a compound of formula IV;
c) reducing the compound of formula IV or the compound of formula III with a suitable reducing agent to provide a compound of formula V;
d) reacting the compound of formula V with a suitable activating agent to provide a compound of formula VI;
e) reacting the compound of formula VI with tetrazole or its salt to provide a compound of formula VII;
f) deprotecting the compound of formula VII, if PG is hydroxy protecting group to provide a compound of formula VIII; and
g) converting the compound of formula VIII into Cenobamate of formula I.

In another aspect, the present invention provides a process for the preparation of cenobamate of formula I, which comprise of reacting the compound of formula VI with tetrazole or a salt thereof, in the presence of a base and a solvent to provide a compound of formula VII.

In another aspect, the present invention provides a process for the purification of Cenobamate of formula I, which comprises dissolving Cenobamate in a suitable solvent and isolating the pure compound by addition of suitable anti-solvent.

In another aspect, the present invention provides crystalline form of Cenobamate characterised by the PXRD pattern shown in FIG. 1, herein designated as crystalline form M.

In another aspect, the present invention provides a process for the preparation of crystalline Cenobamate, which comprises;
a) Dissolving Cenobamate in isopropanol at a suitable temperature;
b) optionally, concentrating the step a) solution partially; and
c) isolating crystalline Cenobamate by adding n-hexane to step a) or step b) reaction mass.

In another aspect, the present invention provides a process for the preparation of Cenobamate of formula I, which comprises:
a) reacting tetrazole with alkylhaloacetate of formula IX in the presence of a base in a suitable solvent to provide a compound of formula X,
b) reacting the compound of formula X with 1-halo-2-chlorobenzene of formula XI in the presence of a suitable organolithium reagent in a suitable solvent to provide a compound of formula XII,
c) converting the compound of formula XII into compound of formula VIII, and
d) converting the compound of formula VIII into Cenobamate of formula I.

In another aspect, the present invention provides an alternative process for the preparation of Cenobamate of formula I, which comprises:
a) reacting tetrazole with alkylhaloacetate of formula IX in the presence of a base in a suitable solvent to provide a compound of formula X;
b) reducing the compound of formula X with a suitable reducing agent in a suitable solvent to provide a compound of formula XIII;
c) oxidizing the compound of formula XIII with a suitable oxidising agent to provide a compound of formula XIV;
d) reacting the compound of formula XIV with 1-halo-2-chlorobenzene of formula XI in the presence of a suitable organolithium reagent in a suitable solvent to provide a compound of formula XV,
e) converting the compound of formula XV into racemic Cenobamate, and
f) resolving the racemic cenobamate obtained in step e) with a suitable resolution agent to provide cenobamate of formula I.

In another aspect, the present invention provides a process for the preparation compound of formula VIII, which comprises:
a) reacting tetrazole with formalin in the presence of a base in a suitable solvent to provide a compound of formula XVI,
b) halogenating the compound of formula XVI with a suitable halogenating agent in a suitable solvent to provide a compound of formula XVII,
c) reacting the compound of formula XVII with 2-chlorobenzaldehyde in the presence of a suitable organolithium reagent in a suitable solvent to provide a compound of formula XV, and
d) resolving the compound of formula XV with a suitable resolution agent to provide a compound of formula VIII.

In another aspect, the present invention provides a process for the preparation of Cenobamate of formula I, which comprises:
a) reacting a compound of formula XVIII with a suitable alcohol in the presence of a catalyst to provide a compound of formula XIX,
b) reacting the compound of formula XIX with Lithium (trimethylsilyl)diazomethane to provide a compound of formula XII,
c) converting the compound of formula XII into cenobamate of compound of formula I.

In another aspect, the present invention provides compounds of formula V, formula VI and formula VII, which are useful in the preparation of Cenobamate of formula I.

In another aspect, the present invention provides use of novel intermediate compounds of formula V, formula VI and formula VII for the preparation of Cenobamate of formula I.

In another aspect, the present invention provides pharmaceutical composition comprising Cenobamate prepared according to the process of the present invention and at least once pharmaceutically acceptable excipient.

### BRIEF DESCRIPTION OF DRAWINGS

FIG. 1 illustrates an X-ray powder diffraction pattern for the Cenobamate crystalline form M of the present invention.

### DETAILED DESCRIPTION OF THE INVENTION

Accordingly, the present invention provides a novel process for the preparation of Cenobamate of formula 1, which is cost effective and commercially viable.

The term “suitable solvent” used in the present invention until unless specified is selected from, but are not limited to “alcoholic solvents” such as methanol, ethanol, isopropyl alcohol, n-propanol, butanol and the like; “ester solvents” such as ethyl acetate, methyl acetate, n-butyl acetate, isobutyl acetate, sec-butyl acetate, isopropyl acetate and the like, “ether solvents” such as tetrahydrofuran, diisoproylether, diethyl ether, methyl tert-butyl ether, dioxane and the like; “hydrocarbon solvents” such as toluene, xylene, cyclohexane, hexane, heptane, n-pentane, petroleum ether and the like; “chloro solvents” such as dichloromethane, ethylene dichloride, carbon tetrachloride, chloroform and the like; “polar aprotic solvents” such as dimethylformamide, dimethylacetamide, dimethylsulfoxide and the like; “nitrile solvents” such as acetonitrile and the like; “ketone solvents” such as acetone, methyl isobutyl ketone, methyl ethylketone and the like; “cyclic amide solvent” such as N-methyl-2-pyrrolidone, 2-piperidinone,azetidin-2-one, N-methyl caprolactame, 1-methylpiperidin-2-one and the like; and water.

The term “reducing agent” used in the present invention until unless specified is selected from, but are not limited to lithium aluminum hydride, sodium borohydride, potassium borohydride, sodium cyanoborohydride, Borane-pyridine complex, sodium hydride, DIBAL-H or sodium bis(2-methyoxyethoxy)aluminumhydride, Pt on alumina, Pd on alumina, Pd/C, Pd(OH₂)—C, Raney Ni, Rh/C, Rh/Al, Pt/C, Ru/C, and PtO₂.

The term “suitable base” used herein the present invention until unless specified is selected from inorganic bases like “alkali metal hydroxides” such as lithium hydroxide, sodium hydroxide, potassium hydroxide and the like; “alkali metal carbonates” such sodium carbonate, potassium carbonate, lithium carbonate and the like; “alkali metal bicarbonates” such as sodium bicarbonate, potassium bicarbonate, lithium bicarbonate and the like; “alkali metal hydrides” such as sodium hydride, potassium hydride, lithium hydride and the like; “alkali metal alkoxides” such as sodium methoxide, sodium ethoxide, sodium tertbutoxide, potassium methoxide, potassium ethoxide, potassium tert-butoxide and the like ammonia and organic bases such as triethylamine, methylamine, ethylamine, 1,8-diazabicycle[5.4.0]undec 7-ene (DBU), 1,5-diazabicyclo(4.3.0) non-5-ene (DBN), lithiumdiisopropylamine (LDA), n-butyllithium, tribenzylamine, isopropylamine, diisopropylamine (DIPA), diisopropylethyl amine (DIPEA), N-methylmorpholine (NMP), N-ethylmorpholine, piperidine, dimethyl aminopyridine (DMAP), morpholine or mixture thereof.

In a first embodiment, the present invention provides a process for the preparation of Cenobamate of formula I,

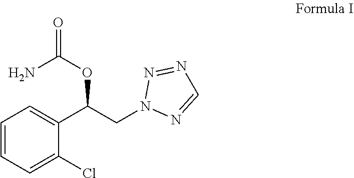 [SMILES: NC(=O)O[C@@H](Cn1ncnn1)c1ccccc1Cl]

which comprises:
a) reacting (R)-(−)-2-Chloromandelic acid of formula II,

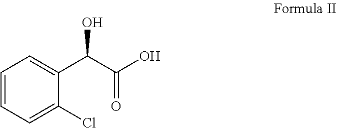 [SMILES: O=C(O)[C@H](O)c1ccccc1Cl]

with a suitable alcohol in the presence of a catalyst to provide a compound of formula III,

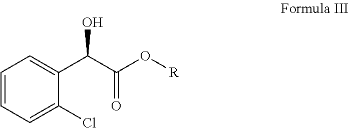

wherein R is C₁ to C₆ alkyl,
b) optionally, reacting the compound of formula III with a suitable hydroxy protecting agent to provide a compound of formula IV,

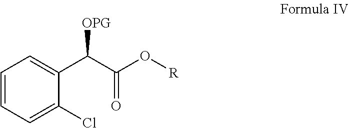

wherein R is C₁ to C₆ alkyl and PG is a hydroxy protecting group,
c) reducing the compound of formula IV or the compound of formula III with a suitable reducing agent to provide a compound of formula V,

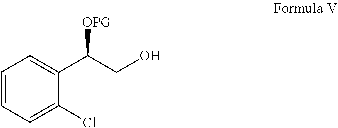

wherein PG is hydrogen or hydroxy protecting group,
d) reacting the compound of formula V with a suitable activating agent to provide a compound of formula VI,

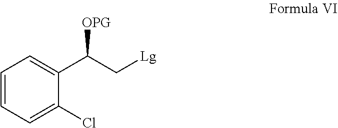

wherein PG is hydrogen or hydroxy protecting group and Lg is leaving group,
e) reacting the compound of formula VI with tetrazole or its salt to provide a compound of formula VII,

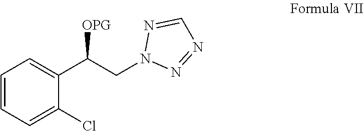

wherein PG is hydrogen or hydroxy protecting group,
f) deprotecting the compound of formula VII, if PG is hydroxy protecting group to provide a compound of formula VIII; and

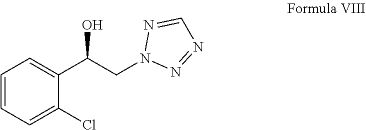 [SMILES: O[C@@H](Cn1ncnn1)c1ccccc1Cl]

g) converting the compound of formula VIII into Cenobamate of formula I.

The starting (R)-(−)-2-Chloromandelic acid of formula II is known in the art and is commercially available. The same can be obtained from available commercial sources.

The step a) of aforementioned process involves the reaction of (R)-(−)-2-chloromandelic acid of formula II with a suitable alcohol selected from but not limited to methanol, ethanol, propanol and the like; preferably methanol; in the presence of a catalyst selected from acids such as hydrochloric acid, sulphuric acid, phosphoric acid and the like; preferably sulphuric acid; under appropriate reaction conditions to provide a compound of formula III, wherein R is alkyl selected from methyl, ethyl, propyl and the like; preferably methyl. The said reaction is carried out at a suitable temperature of about 25° C. to about reflux temperature of the solvent used for a sufficient period of time; preferably for about 2 to 6 hours at about 50° C. to 65° C.

The step b) of aforementioned process involves reacting the compound formula III with suitable hydroxy protecting agent selected from but not limited to acyl halides, aryl halides, pivaloyl halides, trimethylsilylchloride (TMSCI), triethylsilyl chloride, tert-butyl dimethylsilyl chloride (TBDMSCI), tert-butyldiphenylsilyl chloride, (Triisopropylsiloxy) methyl chloride (TOMCl), triisopropylsilylchloride (TIPSCl) and the like; preferably TBDMSCI, to provide a compound of formula IV, wherein R is alkyl selected from methyl, ethyl, propyl and the like; and PG is hydroxy protecting group. The said reaction can be carried out in the presence of a base, preferably imidazole, in a suitable solvent, preferably chloro solvent such as dichloromethane under appropriate reaction conditions to provide compound of formula IV. The step b) reaction is carried out at a suitable temperature of about 0° C. to about 40° C. for a sufficient period of time, preferably for about 6 to 10 hours at about 20° C. to 30° C.

The step c) of aforementioned process involves the reduction of compound of formula IV or the compound of formula III, wherein R is C₁ to C₆ alkyl with a suitable reducing agent selected from sodium borohydride, lithium aluminium hydride, DIBAL-H, sodium cyano borohydride, sodium triacetoxyborohydride and the like; preferably sodiumborohydride, in a suitable solvent, preferably alcoholic solvent such as methanol, under appropriate reaction conditions to provide a compound of formula V, wherein PG is hydrogen or hydroxy protecting group. The step c) reaction is carried out at a suitable temperature of about 0° C. to about 35° C. for sufficient period of time; preferably for about 2 to 4 hours.

The step d) of aforementioned process involves reacting the compound of formula V, wherein in PG is hydrogen or hydroxy protecting group with a suitable activating agent under appropriate reaction conditions to provide a compound of formula VI, wherein PG is hydrogen or hydroxy protecting group; and “Lg” is leaving group selected from halides such as fluoride, chloride, bromide and iodide; sulfonates such as mesylate, tosylate, 4-nitrophenyl sulfonate, trifluoromethyl sulfonate and the like. The suitable activating agent is selected from mesyl halides, tosyl halides, 4-nitrophyenyl sulfonyl halide, phosphorus tribromide, phosphorus trichloride, phosphorus pentachloride, phosphorus pentabromide, carbon tetrachloride and triphenylphospine, thionyl chloride, hydrobromide and the like.

If PG is hydroxy protecting group, then the step d) reaction of compound of formula V with a suitable activating agent is carried out with or without presence of an additional base or catalyst under appropriate reaction conditions to provide compound of formula VI. In a preferred embodiment, the activating agent is halides, and is obtained by the reaction of compound of formula V, wherein PG is TBDMS with carbontetrachloride and triphenyl phosphine in a suitable solvent such as dichloromethane at a suitable temperature of about 20° C. to about 60° C. for a sufficient period of time; preferably for about 2 to 4 hours at about 40-45° C.

If PG is hydrogen, then the step d) reaction of compound of formula V with a suitable activating agent is carried out in the presence of a base and catalyst in a suitable solvent under appropriate reaction conditions to provide a compound of formula VI. In a preferred embodiment, the suitable activating agent is sulfonates, and is obtained by the reaction of compound of formula V, wherein PG is hydrogen with activating agent such as p-toluene sulfonylchloride in the presence of a suitable base such as organic base; preferably triethylamine or diisopropylethylamine; in a suitable solvent selected from nitriles, hydrocarbons, chlorosolvents and/or mixtures thereof; preferably toluene, acetonitrile, dichloromethane or mixtures thereof; in the presence of a dibutyltin oxide catalyst to provide a compound of formula VI, wherein PG is hydrogen and Lg is -OTs. The said reaction is carried out at a suitable temperature from about 10° C. to 30° C. for a sufficient period of time; preferably for about 2 to 4 hrs.

In an embodiment, the compound of formula VI is further crystallized or purified using a suitable solvent selected from hydrocarbons such as toluene, xylene, cyclohexane, hexane, heptane, n-pentane, petroleum ether; and ethers such as tetrahydrofuran, diisoproylether, diethyl ether, methyl tert-butyl ether, dioxane and mixtures thereof; preferably n-hexane, diisopropylether and mixtures thereof.

The step e) of the aforementioned process involves the reaction of compound of formula VI, wherein PG is hydrogen or hydroxy protecting group and Lg is leaving group with tetrazole or a salt thereof, carried out in the presence of a base and a solvent under appropriate reactions conditions to provide a compound of formula VII, wherein PG is hydrogen or hydroxy protecting group. The suitable base is organic or inorganic base; preferably diisopropylethylamine or potassium tertiarybutoxide and the solvent is cyclic amide solvent such as N-methyl-2-pyrrolidone. The said reaction is carried out at a suitable temperature from about 20° C. to about reflux temperature of the solvent used for sufficient period of time; preferably for about 4 to 7 hours at 100° C. to 120° C.

Further, 1N positional isomer of formula VII is also formed along with the formula VII and the same is separated and removed by way of recrystallization or other available purifications methods such as purified using silica gel chromatography preferably purified using silica gel chromatography (Gradient: ethyl acetate in hexane) before proceeding to the next step.

The step f) of aforementioned process involves the deprotection of compound of formula VII, if PG is hydroxy protecting group to provide a compound of formula VIII. The said deprotection reaction is carried out by treating the compound of formula VII with a suitable deprotection agent source selected from acid or base or fluoride ion, palladium-catalyzed hydrogenation and the like, based on the nature of the hydroxy protecting group by the methods known in the art under appropriate reaction conditions to provide the compound of formula VIII. Preferably, the compound of formula VII, wherein PG is TBDMS protecting group is removed by treating it with HCl in THE at a suitable temperature of about 0-40° C. for a time period of time to provide a compound of formula VIII.

The step g) of aforementioned process involves the conversion of compound of formula VIII into Cenobamate of formula I. The reaction introduce carbamoyl group to alcohol compound of formula VIII can be done by known methods in the art such as utilizing 1,1′-carbonyldiimidazole-amine, sodium cyanate-acid, carbamoyl chloride, chlorosulfonyl isocyanate, chlorosulfamoyl isocyanate-water, disuccimidyl carbonate-amine, phosgene-amine, triphosgene-amine, chloroformate-amine, trichloroacetyl chloride-amine, trichloroactylisocyanate, trimethylsilylisocyanate, 1-chlorocarbonyl benzotriazole-amine and the like under appropriate reaction conditions. Preferably, the compound of formula VIII reacts with chlorosulfonyl isocyanate in THE at a suitable temperature of about-20° C. for a time period of 4 to 8 hours to provide Cenobamate of formula I.

In an embodiment, Cenobamate of formula I obtained by the above process further involves purification, which can be carried out by recrystallization of Cenobamate from a suitable solvent or by dissolving Cenobamate in a suitable solvent followed by isolating pure compound by addition of anti-solvent or slurrying the Cenobamate in a suitable solvent or mixtures thereof. The suitable solvent is selected from alcohols, esters, ethers, hydrocarbons, chlorosolvents, water and/or mixtures thereof.

In an embodiment, the intermediate compound obtained in each step of the above process can be used directly for the next reaction as the reaction mixture or as a crude product without isolating as a solid.

In another embodiment, the present invention provides a process for the preparation of Cenobamate of formula I, which comprise of reacting the compound of formula VI, wherein PG is hydrogen or hydroxy protecting group and “Lg” is a leaving group

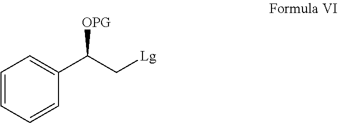

with tetrazole or a salt thereof, in the presence of a base and a solvent to provide a compound of formula VII, wherein PG is hydrogen or hydroxy protecting group.

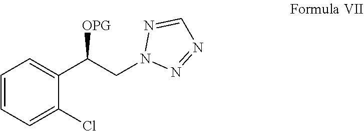

The aforementioned process involves the reaction of the compound of formula VI, wherein “Lg” is leaving group selected from halides such as chloride, bromide and the like; sulfonates such as mesylate, tosylate, 4-nitrophenyl sulfonate, trifluoromethyl sulfonate and the like; with tetrazole or a salt thereof, in the presence of a base and a solvent under appropriate reactions conditions to provide a compound of formula VII. The suitable base is organic or inorganic base; preferably diisopropylethylamine or potassium tertiarybutoxide and the solvent is cyclic amide solvent such as N-methyl-2-pyrrolidone. The said reaction is carried out at a suitable temperature from about 20° C. to about reflux temperature of the solvent used for a sufficient period of time; preferably for about 4 to 7 hours at 100° C. to 120° C.

The compound of formula VII obtained may contaminated with unwanted 1N positional isomers, which can be separated by known techniques such as column purification and crystallization. The use of compound of formula VI of the present invention for the reaction with tetrazole favors the formation of required positional isomer (2N) in comparison to the prior art processes which favors unwanted 1N positional isomers. The compound of formula VII can be further converted into Cenobamate of formula I as per the process described above or by the methods known in the art.

In another embodiment, the present invention provides a process for the purification of Cenobamate of formula I, which comprises a) dissolving Cenobamate of formula I in a suitable solvent; b) adding an anti-solvent to the resulting solution; and c) isolating the pure Cenobamate of formula I. The suitable solvent for dissolution is selected from alcohols, water and mixtures thereof; the suitable anti-solvent is selected from hydrocarbon solvents or mixtures thereof.

In another embodiment, the present invention provides a process for the preparation of crystalline Cenobamate, which comprises;
a) Dissolving Cenobamate in isopropanol at a suitable temperature;
b) optionally, concentrating the step a) solution partially; and
c) isolating crystalline Cenobamate by adding n-hexane to step a) or step b) reaction mass.

The step a) of the aforementioned process involves dissolution of Cenobamate in isopropanol at a suitable temperature of about 25° C. to about reflux temperature; preferably at about 45-50° C.; optionally, the step a) solution was concentrated partially under vacuum; then isolation of crystalline Cenobamate was carried out by addition of n-hexane to step a) or step b) reaction mass. The obtained solid was isolated by filtration, further dried at a suitable temperature under vacuum.

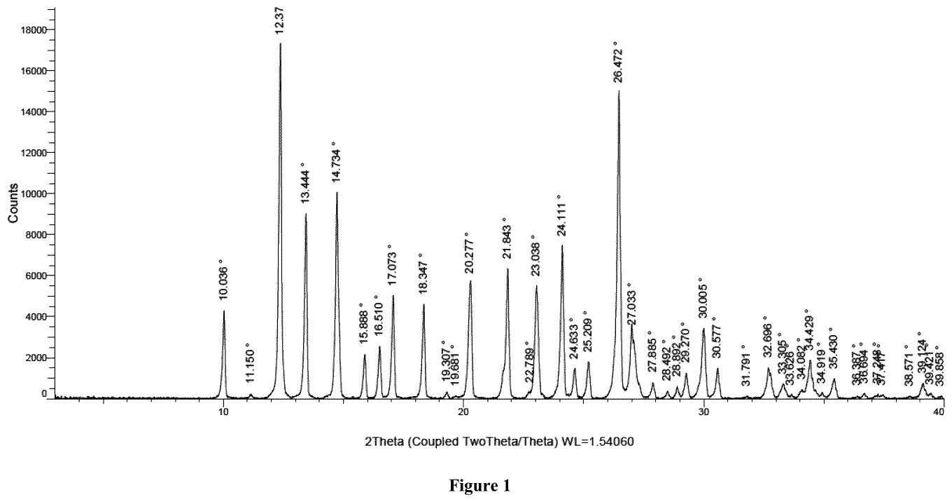

In another embodiment, the present invention provides crystalline form of Cenobamate characterized by the PXRD pattern shown in FIG. 1, herein designated as crystalline form M. The crystalline form M of Cenobamate further characterized by a powder X-ray diffractogram that comprises one or more peaks at 10.03, 12.37, 13.44, 14.73, 17.07, 18.34, 20.27, 21.84, 23.03 24.11, 26.47 and 27.03 degrees 2θ+0.2.

In an embodiment, the Cenobamate obtained by the present invention having chemical purity greater than 99% by HPLC; and having optical purity of greater than 99.9% by HPLC.

In another embodiment, the present invention provides a process for the preparation of Cenobamate of formula I, which comprises:
a) reacting tetrazole with alkylhaloacetate of formula IX, wherein X is halogen; R is alkyl,

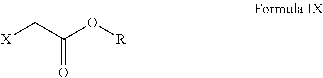

in the presence of base in a suitable solvent to provide a compound of formula X, wherein R is alkyl;

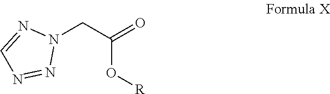

b) reacting the compound of formula X with 1-halo-2-chlorobenzene of formula XI, wherein X is halogen;

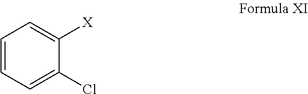

in the presence of a suitable organolithium reagent in a suitable solvent to provide a compound of formula XII; and

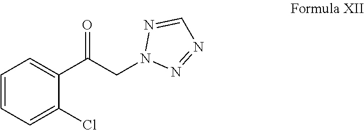 [SMILES: O=C(Cn1ncnn1)c1ccccc1Cl]

c) converting the compound of formula XII into compound of formula VIII, and

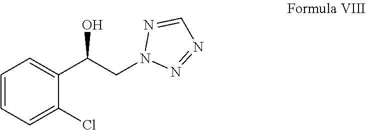 [SMILES: O[C@@H](Cn1ncnn1)c1ccccc1Cl]

d) converting the compound of formula VIII into Cenobamate of formula I.

The step a) of aforementioned process involves the reaction of tetrazole with alkylhaloacetate of formula IX, wherein X is halogen; R is alkyl; in the presence of a suitable base in a suitable solvent under appropriate conditions to provide a compound of formula X, wherein R is alkyl having 1 to 6 carbon atoms.

Further, 1N positional isomer of formula X is also formed along with formula X and the same is separated and removed by way of recrystallisation or other available purifications methods such as purified using silica gel chromatography. Preferably purified using silica gel chromatography (Gradient: ethyl acetate in hexane) before proceeding to the next step.

The step b) of aforementioned process involves the reaction of compound of formula X with 1-halo-2-chlorobenzene of formula XI, wherein X is halogen; in the presence of a suitable organolithium reagent selected from but not limited to iPrMgCl·LiCl, n-butyl lithium, sec-butyllithium, isopropyllithium, tert-butyllithium, phenyl lithium and the like; in a suitable solvent under appropriate conditions to provide a compound of formula XII.

The step c) of aforementioned process involves the conversion of compound of formula XII into compound of formula VIII. The said conversion can be carried out by the methods known in the art such as subjecting an arylketone of formula XII to (R)-selective asymmetric reduction is achieved by biological asymmetric reduction or by chemical asymmetric reduction with a chiral borane reductant, or by asymmetric catalytic hydrogenation, or by asymmetric catalytic transfer hydrogenation to form an alcohol of formula VIII.

The step d) of aforementioned process involves the conversion of compound of formula VIII into cenobamate of compound formula I can be carryout by the methods known in the art.

In another embodiment, the present invention provides an alternative process for the preparation of cenobamate of formula I, which comprises:
a) reacting tetrazole with alkylhaloacetate of formula IX, wherein X is halogen; R is alkyl,

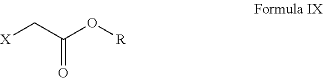

in the presence of a base in a suitable solvent to provide a compound of formula X, wherein R is alkyl,

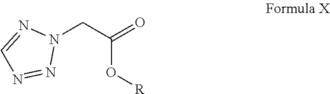

b) reducing the compound of formula X with a suitable reducing agent to provide a compound of formula XIII,

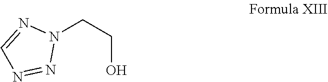

c) oxidizing the compound of formula XIII with a suitable oxidising agent to provide a compound of formula XIV,

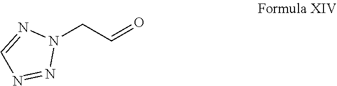

d) reacting the compound of formula XIV with 1-halo-2-chlorobenzene of formula XI in the presence of a suitable organolithium reagent in a suitable solvent to provide a compound of formula XV,

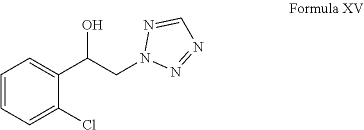 [SMILES: OC(Cn1ncnn1)c1ccccc1Cl]

e) converting the compound of formula XV into racemic cenobamate, and
f) resolving the racemic Cenobamate obtained in step e) with a suitable resolution agent to provide Cenobamate of formula I.

The step a) of aforementioned process involves the reaction of tetrazole with alkylhaloacetate of formula IX, wherein X is halogen; R is alkyl; in the presence of a suitable base in a suitable solvent under appropriate conditions to provide a compound of formula X, wherein R is alkyl. Further, 1N positional isomer of formula X is also formed along with formula X and the same has to be separated and removed by way of recrystallisation or other available purifications methods before proceeding to the next step.

The step b) of aforementioned process involves the reduction of compound of formula X with a suitable reducing agent selected from sodium borohydride, lithium aluminium hydride, DIBAL-H, sodium cyano borohydride, sodium triacetoxyborohydride and the like; in a suitable solvent under appropriate reaction conditions to provide a compound of formula XIII.

The step c) of aforementioned process involves the oxidation of compound of formula XIII with a suitable oxidising agent selected from MnO₂, NaOCl, TEMPO, Jones reagent, Dess-Martin reagent and the like under appropriate conditions to provide a compound of formula XIV.

The step d) of aforementioned process involves the reaction of compound of formula XIV with 1-halo-2-chlorobenzene of formula XI in the presence of a suitable organolithium reagent selected from but not limited to n-butyllithium, sec-butyllithium, isopropyllithium, tert-butyllithium, phenyl lithium and the like; in suitable solvent to provide a compound of formula XV.

The step e) of aforementioned process involves the conversion of compound of formula XV into racemic Cenobamate of formula I and can be carried out by the methods known in the art.

The step f) of aforementioned process involves the resolution of racemic Cenobamate of formula I with a suitable resolution agent selected from chiral acids but not limited to(S)-(+)-camphor-10-sulfonic acid, (1S)-(+)-3-bromocamphor-10-sulfonic acid, (S)-1-phenyl ethane sulphonic acid, (+)-D-di-O-benzoyltartaric acid, (−)-L-di-O-benzoyltartaric acid, (−)-di-O,O′-p-tolyl-L-tartaric acid, (+)-di-O,O′-p-tolyl-D-tartaric acid, R (+)-malic acid, S-(−)-malic acid, (+)-camphanic acid, (−)-camphanic acid, R (−)-1,1′-binaphthalene-2,2′-diyl hydrogen phosphate, S (+)-1,1′-binaphthalene-2,2′-diyl hydrogen phosphate, (+)-camphoric acid, (−)-camphoric acid, S (+)-2-phenylpropionic acid, R (−)-2-phenylpropionic acid, D (−)-mandelic acid, L (+)-mandelic acid, D-tartaric acid, L-tartaric acid and the like under appropriate reaction conditions to provide Cenobamate of formula I.

In another embodiment, the present invention provides a process for the preparation compound of formula VIII,

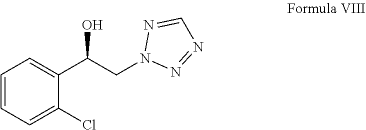 [SMILES: O[C@@H](Cn1ncnn1)c1ccccc1Cl]

which comprises:
a) reacting tetrazole with formalin in the presence of base in a suitable solvent to provide a compound of formula XVI;

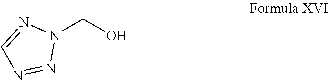

b) halogenating the compound of formula XVI with a suitable halogenating agent in a suitable solvent to provide a compound of formula XVII, wherein X is halogen,

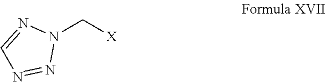

c) reacting the compound of formula XVII with 2-chlorobenzaldehyde in the presence of a suitable organolithium reagent in a suitable solvent to provide compound of formula XV; and

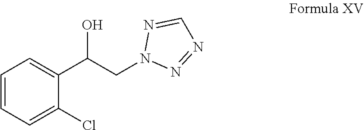 [SMILES: OC(Cn1ncnn1)c1ccccc1Cl]

d) resolving the compound of formula XV with a suitable resolution agent to provide a compound of formula VIII.

The step a) of aforementioned process involves the reaction of tetrazole with formalin in the presence of a base in a suitable solvent under appropriate reaction conditions to provide a compound of formula XVI, which also contains its 1N positional isomer and the same is separated and removed by way of recrystallization or other available purifications methods before proceeding to the next step.

The step b) of aforementioned process involves halogenation of compound of formula XVI with a suitable halogenating agent selected from phosphorus tribromide, phosphorus trichloride, phosphorus pentachloride, phosphorus pentabromide, thionyl chloride, thionyl bromide, HBr and the like; in a suitable solvent under appropriate reaction conditions to provide a compound of formula XVII.

The step c) of aforementioned process involves the reaction of compound of formula XVII with 2-chlorobenzaldehyde in the presence of a suitable organolithium agent selected from but not limited to n-butyllithium, sec-butyllithium, isopropyllithium, tert-butyllithium, phenyl lithium and the like; in a suitable solvent under appropriate conditions to provide a compound of formula XV.

The step c) of aforementioned process involves the resolution of compound of formula XV with a suitable resolution agent as described above for the resolution of formula I, under appropriate conditions to provide a compound of formula VIII.

In another embodiment, the present invention provides a process for the preparation of cenobamate of formula I, which comprises:
a) reacting compound of formula XVIII

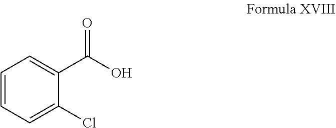

with a suitable alcohol in the presence of a catalyst to provide a compound of formula XIX, wherein R is alkyl,

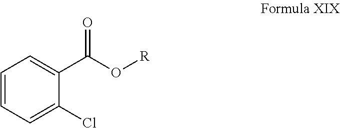

b) reacting the compound of formula XIX with Lithium (trimethylsilyl)diazomethane to provide compound of formula XII, and

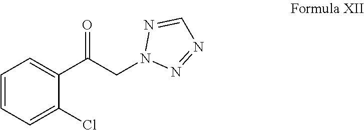 [SMILES: O=C(Cn1ncnn1)c1ccccc1Cl]

c) converting the compound of formula XII into cenobamate of formula I.

The step a) of aforementioned process involves the reaction of compound of formula XVIII in the presence of a suitable alcohol selected from but not limited to methanol, ethanol, propanol and the like; in the presence of a catalyst selected from acids such as hydrochloric acid, sulphuric acid, phosphoric acid and the like under appropriate reaction conditions to provide a compound of formula XIX, wherein R is alkyl selected from methyl, ethyl, propyl and the like.

The step b) of aforementioned process involves the reaction of compound of formula XIX with Lithium (trimethylsilyl)diazomethane to provide a compound of formula XII. The reaction can be carried out in presence or absence of a base in a suitable solvent under appropriate reaction conditions. The said reaction directly provides the required compound of formula XII without formation additional 1N-positional isomer and thereby avoiding tedious column purifications/recrystallisation in order to isolate and separate the 1N position isomer from the required 2N positional isomer compound as in the prior art.

The step c) of aforementioned process involves conversion of compound of formula XII into cenobamate of formula I as described in step c) & step d) of the second embodiment of the present invention or by the methods known in the art.

In a further embodiment, the present invention provides compounds of formula V, formula VI and formula VII represented by the following structural formula, which are useful in the preparation of Cenobamate of formula I.

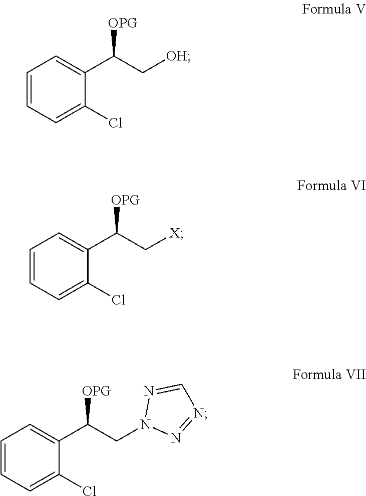

wherein PG is a hydroxy protecting group and X is halogen

In a preferred embodiment, the present invention provides compounds of formula Vb, formula VIb and formula VIIb represented by the following structural formula, which are useful in the preparation of Cenobamate of formula I.

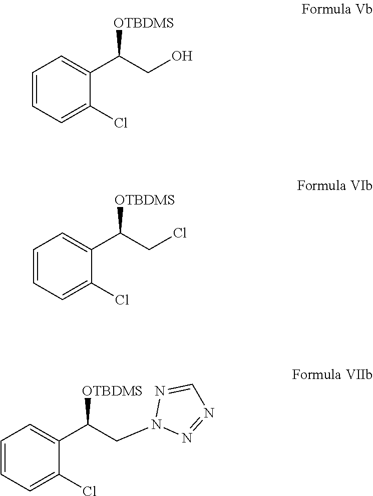 [SMILES: CC(C)(C)[Si](C)(C)O[C@@H](CO)c1ccccc1Cl ; CC(C)(C)[Si](C)(C)O[C@@H](CCl)c1ccccc1Cl ; CC(C)(C)[Si](C)(C)O[C@@H](Cn1ncnn1)c1ccccc1Cl]

wherein TBDMS is tert-butyldimethylsilyl group.

In a further embodiment, the present invention provides use of compounds of formula V, formula VI, formula VII, formula Vb, formula VIb and formula VIIb as an intermediate for the preparation of Cenobamate of formula I.

In another embodiment, the present invention provides a process for the preparation of (2R)-2-(2-chlorophenyl) oxirane, which comprises of the following steps;
a) esterification of (R)-(−)-2-Chloromandelic acid with a suitable alcohol such as methanol, ethanol, isopropanol and the like; in the presence of a suitable acid catalyst such as hydrochloric acid, sulphuric acid and the like; to provide a corresponding alkyl (2R)-(2-chlorophenyl) (hydroxy)acetate;
b) reducing the alkyl (2R)-(2-chlorophenyl) (hydroxy)acetate with a suitable reducing agent such as sodium borohydride, sodiumcyanoborohydride and the like in a suitable solvent; to provide a (1R)-1-(2-chlorophenyl) ethane-1,2-diol; and
c) reacting the (1R)-1-(2-chlorophenyl) ethane-1,2-diol with trimethylorthoformate and trimethylsilylchloride in a suitable solvent followed by treating with a suitable base to provide (2R)-2-(2-chlorophenyl) oxirane.

In a further embodiment, the present invention provides pharmaceutical composition comprising Cenobamate prepared according to the process of the present invention and at least once pharmaceutically acceptable excipient.

The process details of the invention are provided in the examples given below, which are provided by way of illustration only and therefore should not be construed to limit the scope of the invention.

### EXAMPLES

### Example 1: Preparation of (R)-methyl 2-(2-chlorophenyl)-2-hydroxyacetate (Formula III, Wherein R is Methyl)

To a solution of (R)-2-(2-chlorophenyl)-2-hydroxyacetic acid (100 g) in methanol (1 L), sulfuric acid (20 ml) was added at room temperature. The reaction mass was heated to 60-65° C. and stirred for 2 hours. After completion of the reaction, the reaction mass was cooled to 40-45° C. and distilled off under vacuum at 40-45° C. Water (400 ml) and ethyl acetate (600 ml) was added to the obtained crude at 25-35° C. and stirred for 15 mins. The aqueous and organic layers were separated, the aqueous layer was extracted with ethyl acetate; the organic layer was washed with sodium bicarbonate solution followed by sodium chloride solution, dried over sodium sulfite and distilled off under vacuum at below 45° C. to get the title compound. Yield: 107 g.

### Example 2: Preparation of (R)-1-(2-chlorophenyl) ethane-1,2-diol (Formula V, Wherein PG is Hydrogen)

(R)-methyl 2-(2-chlorophenyl)-2-hydroxyacetate (107 g) was dissolved in methanol (750 ml) at room temperature and cooled to 0-5° C. Sodium borohydride (37.46 g) was added to the reaction mass at 0-5° C., temperature raised to 25-35° C. and stirred for 3 hours. After completion of the reaction, the reaction mass was distilled off under vacuum at below 50° C. Water (400 ml) and Ethyl acetate (600 ml) was added to the obtained crude at 25-35° C. and stirred for 10 mins. The aqueous and organic layers were separated, the aqueous layer was extracted with ethyl acetate; the organic layer was washed with sodium bicarbonate solution followed by sodium chloride solution, dried over sodium sulfite and distilled off under vacuum below 45° C. to get the title compound. Yield: 88 g.

### Example 3: Preparation of (R)-2-(2-chlorophenyl)-2-hydroxyethyl 4-methyl benzenesulfonate (Formula VI, Wherein PG is Hydrogen and Lg is p-Toluenesulfonyl)

To a mixture of (R)-1-(2-chlorophenyl) ethane-1,2-diol (91 g) in dichloromethane (1.3 L), dibutyltin oxide (2.6 g) and p-toluenesulfonyl chloride (90 g) was added at room temperature. The reaction mass was cooled to 10-15° C. and triethylamine (64.2 g) was added to it at same temperature. Then the reaction was temperature raised to room temperature and stirred for 2 hours. After completion of the reaction, water (500 ml) was added to the reaction mass and stirred for 10 mins. The aqueous and organic layers were separated, the aqueous layer was extracted with dichloromethane; the organic layer was washed with sodium bicarbonate solution followed by sodium chloride solution, dried over sodium sulfite and distilled off under vacuum below 45° C. to get the title compound.

### Example-4: Preparation of (R)-2-(2-chlorophenyl)-2-hydroxyethyl 4-methyl benzenesulfonate (Formula VI, wherein PG is hydrogen and Lg is p-toluenesulfonyl)

The title compound obtained in a similar manner to example-3 using diisopropylethylamine in place of triethylamine and using mixture of toluene and acetonitrile as a solvent instead of dichloromethane. The obtained compound further purified from mixture of n-hexane and diisopropylether. Purity by HPLC: 97%.

### Example 5: Preparation of (R)-1-(2-chlorophenyl)-2-(2H-tetrazol-2-yl) ethanol (Formula VIII)

A solution of (R)-2-(2-chlorophenyl)-2-hydroxyethyl 4-methylbenzenesulfonate (141 g) in N-methyl-2-pyrrolidone was added to a mixture of tetrazole (60.4 g), diisopropyl ethylamine (166.7 g) and N-methyl-2-pyrrolidone (564 ml) at room temperature. The reaction mass was heated to 110° C. and stirred for 6 hours at the same temperature. After completion of the reaction, the reaction mass was cooled to room temperature; water (500 ml) and ethyl acetate (600 ml) was added to it. The aqueous and organic layers were separated, the aqueous layer was extracted with ethyl acetate; the organic layer was washed with sodium bicarbonate solution followed by sodium chloride solution, dried over sodium sulfite and distilled off under vacuum at below 45° C. The obtained crude was purified by column chromatography using DCM/MTBE to get the title compound. Yield: 40 g.

### Example 6: Preparation of (R)-1-(2-chlorophenyl)-2-(2H-tetrazol-2-yl) ethanol (Formula VIII)

The title compound obtained in a similar manner to example 5 using potassium tertiarybutoxide in place of diisopropylethylamine and final column purification was carried out using ethyl acetate/hexane.

### Example 7: Preparation of Cenobamate (Formula I)

(R)-1-(2-chlorophenyl)-2-(2H-tetrazol-2-yl) ethanol (VII) (100 g) was dissolved in tetrahydrofuran (800 ml) under nitrogen atmosphere. The reaction mass was cooled to −30 to −40° C. using dry ice and acetone bath. Chlorosulfonyl isocyanate (126 g) was added to the reaction mass and stirred for 4 hours at −30 to −40° C. After completion of the reaction, water (100 ml) was added to the reaction mass at −30 to −40° C.; and the reaction mass temperature was raised to 25-35° C. then distilled off under vacuum at below 50° C. Water (400 ml) and ethyl acetate (500 ml) was added to the reaction mass at 25-5° C. and stirred for 15 mins. The aqueous and organic layers were separated, the aqueous layer was extracted with ethyl acetate; the organic layer was washed with sodium chloride solution, dried over sodium sulfite and distilled off under vacuum below 45° C. The obtained crude was dissolved in isopropyl alcohol and n-hexane was added to it and stirred at 25-35° C. The obtained solid was filtered, washed with n-hexane and dried to get the title compound. Yield: 100 g.

### Example 8: Purification of Cenobamate (Formula I)

Cenobamate (50 g) was dissolved in isopropyl alcohol (250 ml) at 45-50° C. The reaction mass was subjected to carbon treatment, filtered through hyflow and washed with isopropyl alcohol at 45-50° C. The filtrate was distilled off partially up to 1 volume and hexane (250 ml) was added to it. Then the reaction mass was distilled off under vacuum. Mixture of isopropyl alcohol and n-hexane was added to the obtained crude at 25-35° C., stirred and the solid obtained was filtered, washed with isopropyl alcohol and then dried to get the pure Cenobamate. Yield: 40 g; Purity by HPLC: 99.3%; Chiral purity: 99.9% The PXRD pattern of the obtained Cenobamate is shown in FIG. 1.

### Example 9: Preparation of methyl (R)-2-((tert-butyldimethylsilyl)oxy)-2-(2-chloro phenyl)acetate (Formula IV, Wherein R is Methyl and PG is TBDMS)

To a clean and round RBF, methyl (R)-2-(2-chlorophenyl)-2-hydroxyacetate (15 g), imidazole (6.6 g) and dichloromethane (100 ml) were added and cooled to 0-5° C. To this reaction mass, tert-butyldimethylsilyl chloride (12.3 g) in dichloromethane (50 ml) was added at 0-5° C. Then the reaction mixture was warmed to room temperature and stirred for 8 h. After completion of the reaction, the reaction mass was washed with water followed by brine solution and then distilled off under vacuum to get the title compound.

### Example 10: Preparation of (R)-2-((tert-butyldimethylsilyl)oxy)-2-(2-chlorophenyl) ethan-1-ol (Formula V, wherein PG is TBDMS)

To a solution of methyl (R)-2-((tert-butyldimethylsilyl)oxy)-2-(2-chlorophenyl)acetate (15 g) in methanol (90 ml), sodium borohydride (2.16 g) was added in portion at a 0-5° C. and stirred for 2 h. After completion of the reaction, pH of the reaction mass was adjusted to 5 with acetic acid. The solvent from the reaction mass was evaporated under reduced pressure; water (100 ml) was added and extracted into ethyl acetate. The extracted ethyl acetate layer was washed with 5% sodium bicarbonate solution followed by brine solution and then distilled off under vacuum to get the title compound.

### Example 11: Preparation of (R)-tert-butyl (2-chloro-1-(2-chlorophenyl) ethoxy) dimethylsilane (Formula VI, Wherein Lg is Chloro and PG is TBDMS)

To a solution of (R)-2-((tert-butyldimethylsilyl)oxy)-2-(2-chlorophenyl) ethan-1-ol (10 g) in dichloromethane (60 ml), carbon tetrachloride (13.5 ml) and triphenylphosphine (13.7 g) were added at room temperature. Then the reaction mass was heated to 45° C., stirred for 2 h at the same temperature and the solvent was evaporated under reduced pressure. n-hexane (100 ml) was added to the obtained crude, filtered and the filtrate was concentrated to get the title compound.

### Example 12: Preparation of (R)-2-(2-((tert-butyldimethylsilyl)oxy)-2-(2-chlorophenyl) ethyl)-2H-tetrazole (Formula VII, wherein PG is TBDMS)

To a solution of 1H-tetrazole (1.65 g) in N-methyl-2-pyrrolidone (20 ml), potassium t-butoxide (2.24 g) was added at 0-5° C. and stirred for 30 mins at the same temperature. (R)-tert-butyl (2-chloro-1-(2-chlorophenyl) ethoxy)dimethylsilane (6 g) in N-methyl-2-pyrrolidone (10 ml) was slowly added to the above reaction mass at room temperature. The reaction mass was heated to 110° C. and stirred for 6 h. After completion of the reaction, the reaction mass was poured into ice cold water and extracted with ethyl acetate. The extracted ethyl acetate layer was washed with water followed by saturated aqueous sodium chloride solution, dried over sodium sulfate and concentrated to get oily residue. The residue was further purified using silica gel chromatography (Gradient: 20% to 50% ethyl acetate in hexane) to get the title compound.

### Example 13: Preparation of (R)-1-(2-chlorophenyl)-2-(2H-tetrazol-2-yl) ethan-1-ol (Formula VIII)

A solution of (R)-2-(2-((tert-butyldimethylsilyl)oxy)-2-(2-chlorophenyl)ethyl)-2H-tetrazole (2 g) in THF (10 ml) was cooled to 0-5° C. and hydrochloric acid (10 ml) was added to it at the same temperature. The reaction mass temperature was adjusted to room temperature and stirred for 8 h. After completion of reaction, pH of the reaction mass was adjusted to 7-7.5 with saturated sodium bicarbonate solution and extracted into ethyl acetate. The extracted ethyl acetate layer dried over sodium sulfate and then concentrated to get the title compound.

### Example 14: Preparation of (R)-1-(2-chlorophenyl)-2-(2H-tetrazol-2-yl)ethyl carbamate (Cenobamate, Formula I)

To a clean and dry RBF, (R)-1-(2-chlorophenyl)-2-(2H-tetrazol-2-yl) ethan-1-ol (1 g) and THF (15 ml) was added and cooled to −20° C. To this, chlorosulfonyl isocyanate (0.46 ml) was added drop wise at −20° C. and stirred for 2 h at the same temperature. After completion of the reaction, water was added to the reaction mass and neutralized with 5% sodium bicarbonate solution. The obtained solid was filtered, washed with THF and dried under vacuum at 50° C. for 6 h to get the title compound.

Yield: 1 g. Purity by HPLC: 99.95%

What is claimed:

1. A process for the preparation of Cenobamate of 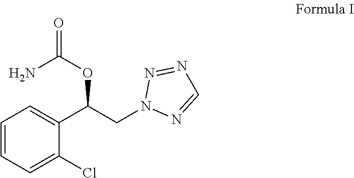 [SMILES: NC(=O)O[C@@H](Cn1ncnn1)c1ccccc1Cl]
which comprises:
a) reacting (R)-(−)-2-Chloromandelic acid of formula II, 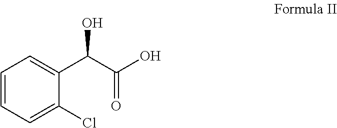 [SMILES: O=C(O)[C@H](O)c1ccccc1Cl]
with a suitable alcohol to provide a compound of formula III, 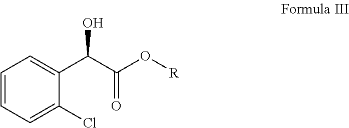
wherein R is C₁ to C₆ alkyl,
b) optionally, reacting the compound of formula III with a suitable hydroxy protecting agent to provide a compound of formula IV, 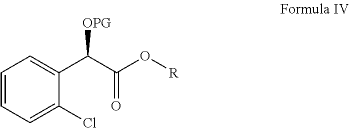
wherein R is C₁ to C₆ alkyl and PG is a hydroxy protecting group,
c) reducing the compound of formula IV or the compound of formula III with a suitable reducing agent to provide a compound of formula V, 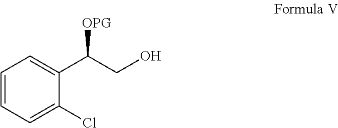
wherein PG is hydrogen or hydroxy protecting group,
d) reacting the compound of formula V with a suitable activating agent to provide a compound of formula VI, 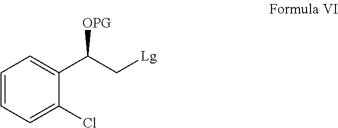
wherein PG is hydrogen or hydroxy protecting group and Lg is leaving group,
e) reacting the compound of formula VI with tetrazole or its salt to provide a compound of formula VII, 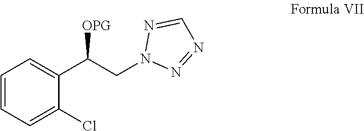
wherein PG is hydrogen or hydroxy protecting group,
f) deprotecting the compound of formula VII, if PG is hydroxy protecting group to provide a compound of formula VIII; and 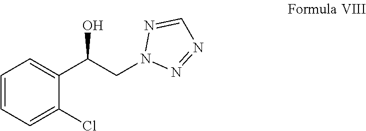 [SMILES: O[C@@H](Cn1ncnn1)c1ccccc1Cl]
g) converting the compound of formula VIII into Cenobamate of formula 1.

2. The process as claimed in claim 1, wherein the suitable alcohol in step a) is selected from methanol, ethanol, propanol, isopropanol and butanol.

3. The process as claimed in claim 1, wherein the step a) reaction is carried out in the presence of a catalyst selected from hydrochloric acid, sulphuric acid, phosphoric acid or mixtures thereof.

4. The process as claimed in claim 1, wherein the suitable hydroxy protecting agent in step b) comprises acyl halides, aryl halides, pivaloyl halides, trimethylsilylchloride (TMSCI), triethylsilyl chloride, tert-butyldimethylsilyl chloride (TBDMSCI), tert-butyldiphenyl silyl chloride, (Triisopropylsiloxy)methyl chloride (TOMCl) or triisopropylsilylchloride (TIPSCl).

5. The process as claimed in claim 1, wherein the suitable reducing agent in step c) comprises sodium borohydride, lithium aluminium hydride, DIBAL-H, sodium cyano borohydride, sodium triacetoxyborohydride and/or mixtures thereof.

6. The process as claimed in claim 1, wherein the suitable activating agent in step d) is selected from mesyl halides, tosyl halides, 4-nitrophyenyl sulfonyl halide, phosphorus tribromide, phosphorus trichloride, phosphorus pentachloride, phosphorus pentabromide, carbon tetrachloride and triphenylphospine, thionyl chloride and hydrobromide.

7. The process as claimed in claim 1, wherein the step e) reaction is carried out in the presence of a base and a solvent.

8. The process as claimed in claim 7, wherein the base is diisopropylethylamine or potassium tertiarybutoxide and the solvent is N-methyl-2-pyrrolidone.

9. The process as claimed in claim 1, wherein the step f) deprotection is carried out using an acid or base or fluoride ion, which comprises HCL, H₂SO₄, HF, methanolic HCL p-toluenesulfonic acid, trifluoromethanesulfonic acid, aq. NaOH, aq·KOH, sodium carbonate, potassium carbonate or tetra-n-butylammonium fluoride.

10. The process as claimed in claim 1, wherein in step g) the conversion of compound of formula VIII into Cenobamate of formula 1 is carried out by reacting the compound of formula VIII with chlorosulfonyl isocyanate in a suitable solvent.

11. A process for the preparation of Cenobamate of formula I, which comprise of reacting the compound of formula VI, wherein PG is hydrogen or hydroxy protecting group and “Lg” is a leaving group 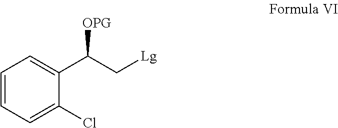
with tetrazole or a salt thereof, in the presence of a base and a solvent to provide a compound of formula VII, 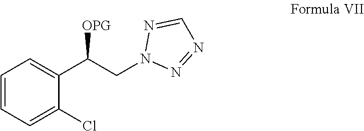
wherein PG is hydrogen or hydroxy protecting group.

12. The process as claimed in claim 11, wherein the leaving group is selected from chloride, bromide, iodide, fluoride, mesylate, tosylate, 4-nitrophenyl sulfonate and trifluoromethyl sulfonate.

13. The process as claimed in claim 11, wherein the base is selected from diisopropylethylamine or potassium tertiarybutoxide and the solvent is N-methyl-2-pyrrolidone.

14. A process for the purification of Cenobamate of formula I, which comprises
a) dissolving Cenobamate of formula I in a suitable solvent;
b) adding an anti-solvent to the resulting solution; and
c) isolating the pure Cenobamate of formula I.

15. The process as claimed in claim 14, wherein the suitable solvent in step a) is selected from alcohols such as methanol, ethanol, isopropanol and butanol; and the anti-solvent is selected from hydrocarbons such as hexane, heptanes, cyclohexane and mixtures thereof.

16. A crystalline form M of Cenobamate characterized by a powder X-ray diffractogram substantially as shown in FIG. 1.

17. A compound of formulae selected from: 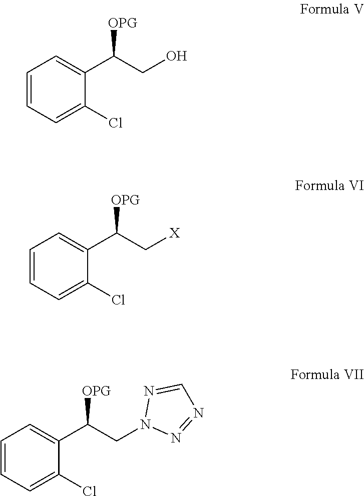
wherein PG is a hydroxy protecting group and X is a leaving group.

18. The compound as claimed in claim 17 are selected from: 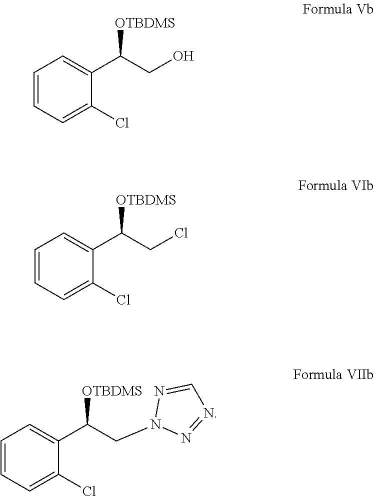 [SMILES: CC(C)(C)[Si](C)(C)O[C@@H](CO)c1ccccc1Cl ; CC(C)(C)[Si](C)(C)O[C@@H](CCl)c1ccccc1Cl ; CC(C)(C)[Si](C)(C)O[C@@H](Cn1ncnn1)c1ccccc1Cl]
wherein TBDMS is tert-butyldimethylsilyl group.

19. The use of the compounds as claimed in claim 17 as an intermediate for the preparation of Centobamate of Formula I.

20. The use of the compounds as claimed in claim 18 as an intermediate for the preparation of Centobamate of Formula I.
